(19) United States
(12) Patent Application Publication
(10) Pub. No.: US 2023/0355295 A1
(43) Pub. Date: Nov. 9, 2023

(54) ELECTROSURGICAL SYSTEMS AND METHODS FOR TIME DOMAIN REFLECTOMETRY BASED TISSUE SENSING

(71) Applicant: Covidien LP, Mansfield, MA (US)

(72) Inventor: Richard L. Croft, Mead, CO (US)

(21) Appl. No.: 17/739,361

(22) Filed: May 9, 2022

(51) Int. Cl. A61B 18/14

(52) U.S. Cl. CPC A61B 18/1445; A61B 2018/0063

(57) ABSTRACT

A method of sealing tissue in accordance with the present disclosure include grasping tissue between first and second jaw members, applying a signal to the grasped tissue in anticipation of tissue treatment, based on a tissue sense algorithm, receiving a reflected signal from the first and second jaw members, and determining a tissue property based on the reflected signal. Electrosurgical systems configured to implement the method are also provided.

### FIELD

The present disclosure relates to electrosurgery and, more particularly, to electrosurgical systems and methods for treating tissue.

### BACKGROUND

In bipolar electrosurgery, electrical current is conducted through tissue positioned between electrodes of different polarity to heat and thereby treat the tissue. Bipolar electrosurgery often involves the use of an electrosurgical forceps, a pliers-like instrument that relies on mechanical action between its jaws to grasp, clamp, and constrict tissue. Electrosurgical forceps, more specifically, utilize mechanical clamping action and electrical energy to treat, e.g., cauterize, coagulate, and/or seal, clamped tissue.

Whereas cauterization involves the use of heat to destroy tissue and coagulation is a process of desiccating tissue such that the tissue cells are ruptured and dried, tissue sealing is a process of liquefying the collagen, elastin, and ground substances in the tissue so that they reform into a fused mass with significantly reduced demarcation between opposing tissue structures. In order to create an effective tissue seal, two predominant mechanical parameters must be accurately controlled: the pressure applied to the tissue and the gap distance between the electrodes. In addition, electrosurgical energy must be applied to the tissue under controlled conditions, e.g., controlling the intensity, frequency, and duration of electrosurgical energy application to tissue, to ensure creation of an effective tissue seal.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

A method of treating tissue in accordance with the present disclosure includes grasping tissue between first and second jaw members, applying a signal to the grasped tissue in anticipation of tissue treatment based on a tissue sense algorithm, receive a reflected signal from the first and second jaw members at a plurality of frequencies, and determining a tissue property or a condition at the first and second jaw members based on the reflected signal.

In an aspect of the present disclosure, the method may further include adjusting a tissue sealing algorithm based on the determined tissue property.

In another aspect of the present disclosure, the method may further include applying energy to the grasped tissue in accordance with a tissue sealing algorithm to seal the grasped tissue, wherein the tissue sealing algorithm is independent of the tissue sense algorithm.

In yet another aspect of the present disclosure, the tissue sense algorithm may be controlled in a first manner, and the tissue sealing algorithm is controlled in a second, different manner.

In still another aspect of the present disclosure, a delay time and/or a voltage of the reflected signal may at the plurality of frequencies is utilized to determine the tissue property.

In still yet another aspect of the present disclosure, the tissue property is impedance at each of the plurality of frequencies.

In an aspect of the present disclosure, at least a portion of the tissue sealing algorithm may adjust energy output to track an impedance versus time trajectory.

In another aspect of the present disclosure, the condition at the first and second jaw members may be one of an open circuit condition or a short circuit condition.

In yet another aspect of the present disclosure, determining the tissue property may include determining a frequency content of the reflected signal and determining a tissue property based on the determined frequency content of the reflected signal.

In still another aspect of the present disclosure, the method may further include after applying energy to the grasped tissue in accordance with the tissue sense algorithm and before applying the energy to the grasped tissue in accordance with the tissue sealing algorithm, implementing a delay period where no energy is applied.

An electrosurgical system for treating tissue in accordance with the present disclosure includes electrosurgical forceps including first and second jaw members and an electrosurgical generator. The electrosurgical generator includes a processor and a memory. The memory includes instructions stored thereon, which, when executed by the processor cause the system to: apply a signal to tissue that is grasped between the first and second jaw members in anticipation of tissue treatment, based on a tissue sense algorithm; receive a reflected signal from the first and second jaw members at a plurality of frequencies; and determine a tissue property or a condition at the first and second jaw members based on the reflected signal.

In an aspect of the present disclosure, the instructions, when executed by the processor, may further cause the system to adjust a tissue sealing algorithm based on the determined tissue property.

In another aspect of the present disclosure, the instructions, when executed by the processor, may further cause the system to apply energy to the grasped tissue in accordance with a tissue sealing algorithm to seal the grasped tissue, wherein the tissue sealing algorithm is independent of the tissue sense algorithm.

In yet another aspect of the present disclosure, the instructions, when executed by the processor, may further cause the system to control the pre-treatment algorithm in a first manner, and the tissue sealing algorithm is controlled in a second, different manner.

In still another aspect of the present disclosure, a delay time and/or a voltage of the reflected signal at the plurality of frequencies may be utilized to determine the tissue property.

In an aspect of the present disclosure, the tissue property may be impedance at each of the plurality of frequencies.

In another aspect of the present disclosure, at least a portion of the tissue sealing algorithm may adjust energy output to track an impedance versus time trajectory.

In yet another aspect of the present disclosure, the condition at the first and second jaw members may be one of an open circuit condition or a short circuit condition.

In still another aspect of the present disclosure, when determining the tissue property, the instructions, when executed by the processor, may further cause the system to determine a frequency content of the reflected signal and determine a tissue property based on the determined frequency content of the reflected signal.

A non-transitory computer-readable medium storing instructions which, when executed by a processor, cause the processor to perform a computer-implemented method of treating tissue, includes grasping tissue between first and second jaw members, applying a signal to the grasped tissue in anticipation of tissue treatment based on a tissue sense algorithm, receive a reflected signal from the first and second jaw members at a plurality of frequencies, and determining a tissue property or a condition at the first and second jaw members based on the reflected signal.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an electrosurgical system in accordance with the present disclosure, including an electrosurgical forceps and an electrosurgical generator;

FIGS. 2A and 2B are enlarged, perspective views of a distal end portion of the forceps of FIG. 1 with an end effector assembly thereof disposed in spaced-apart and approximated positions, respectively;

FIG. 3 is a schematic illustration of a robotic surgical system configured for use in accordance with the present disclosure;

FIG. 4 is a block diagram of the generator of FIG. 1;

FIG. 5 is a schematic illustration showing the electrosurgical generator of FIG. 1 connected to the forceps of FIG. 1 with tissue grasped by jaw members of the end effector assembly of the forceps;

FIG. 6 is a flow diagram illustrating a method of treating tissue in accordance with the present disclosure;

FIG. 7 illustrates the results of a simulation of a signal communicated from generator of FIG. 1 to the end effector assembly of the forceps of FIG. 1 and a measured reflected signal at the generator;

FIG. 8 is a plot of an oscilloscope trace showing a transmitted signal and a reflected signal in the system of FIG. 1 with the jaw members of the end effector assembly of the forceps of FIG. 1 in an open position and not grasping tissue;

FIG. 9 is a plot of an oscilloscope trace showing a transmitted pulse and a reflected pulse in the system of FIG. 1 wherein an impedance between the jaw members of the end effector assembly of the forceps of FIG. 1 is approximately 100 ohms;

FIG. 10 is a simulation of an ideal reflected pulse from a 100 ohm load using the system of FIG. 1;

FIG. 11 is a plot of an oscilloscope trace illustrating the transmitted signal and reflected signal in the system of FIG. 1 wherein an impedance between the jaw members of the end effector assembly of the forceps of FIG. 1 is approximately 50 ohms;

FIG. 12 is a plot showing the frequency spectrum with a short circuit between the jaw members of forceps of FIG. 1;

FIG. 13 is a plot showing the frequency spectrum with an open circuit between the jaw members of forceps of FIG. 1;

FIG. 14 is a plot showing the frequency spectrum with the jaw members of forceps of FIG. 1 wherein an impedance between the jaw members is approximately 50 ohms; and

FIG. 15 is a table showing amplitudes at different frequencies of the return signal, for the loads shown in FIGS. 12-14.

### DETAILED DESCRIPTION

This disclosure enables sensing of characteristics of tissue (and/or other matter) grasped between jaw members of an electrosurgical forceps using pulsed time domain reflectometry. Determining the characteristics of tissue (and/or other matter) in this manner provides the benefits of being fast and not needing additional sensing electrodes. A single short pulse contains a broad band (many) frequencies in the single pulse. By looking at the amplitude and phase of these signals, more information is available within a single pulse.

A sweep of individual frequency over the same bandwidth as the frequencies in the pulse takes time. However, a single pulse with all the frequency content may be sent and processed much faster than a sweep of individual frequencies covering the same range. Using a single short pulse provides the additional benefit of not needing additional sense electrodes because the reflection is returned from the same electrodes being used for energy delivery. Such pulses in accordance with this disclosure may be applied before, during (at random, intermittently, in response to user input, in response to sensed feedback, based on a status or phase of tissue treatment, in between energy deliveries, etc.), and/or after energy delivery.

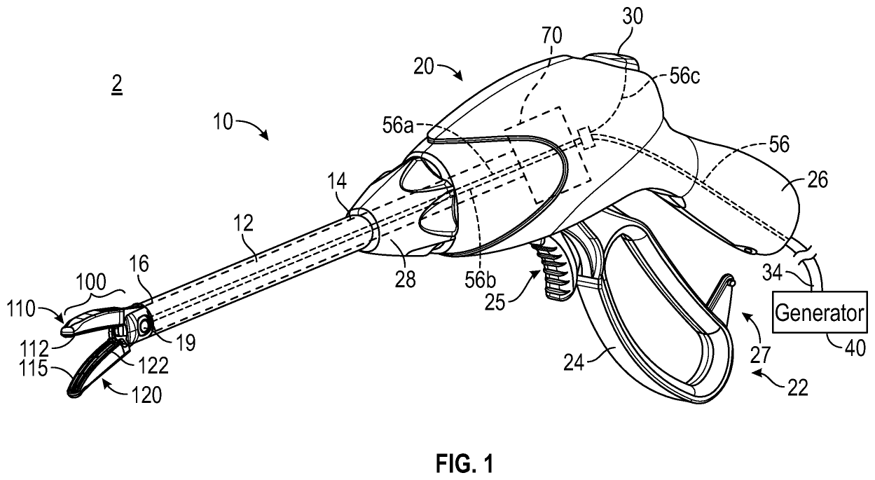

Referring to FIG. 1, an electrosurgical system in accordance with the present disclosure is shown generally identified by reference numeral 2. Electrosurgical system 2 includes an electrosurgical forceps 10 and an electrosurgical generator 40. Electrosurgical forceps 10 is shown and described herein as a shaft-based, manual device. However, any other suitable electrosurgical forceps, whether shaft-based, hemostat-style, manual, partly powered, fully powered, robotic, etc. may be utilized in accordance with the present disclosure. Obviously, different connections and considerations apply to each particular type of instrument; however, the aspects and features of the present disclosure with respect to treating tissue remain generally consistent with respect to any suitable instrument.

Continuing with reference to FIG. 1, forceps 10 includes a shaft 12, a housing 20, a handle assembly 22, a trigger assembly 25, a rotating assembly 28, and an end effector assembly 100. Shaft 12 has a distal end portion 16 configured to mechanically engage end effector assembly 100 and a proximal end portion 14 that mechanically engages housing 20. A cable 34 couples forceps 10 to electrosurgical generator 40 for transmitting energy and control signals between generator 40 and forceps 10. Cable 34 houses a plurality of wires 56 that are internally divided within handle assembly 22 and/or in shaft 12 into wires 56a-56c, which electrically interconnect end effector assembly 100, activation switch 30, and/or generator 40 with one another.

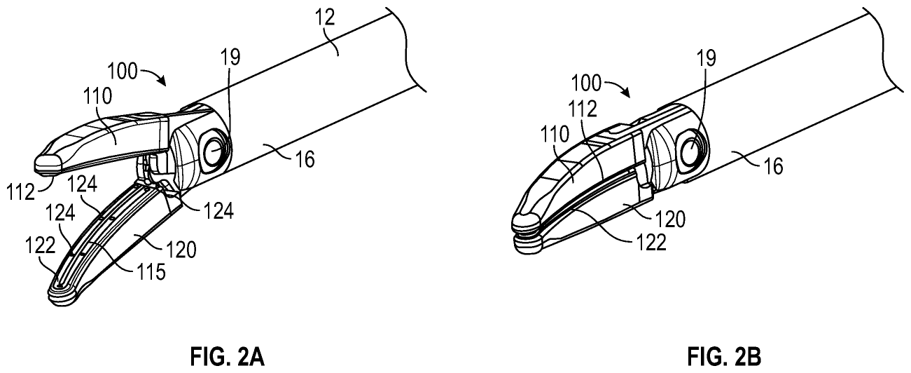

Handle assembly 22 includes a movable handle 24 and a fixed handle 26. Fixed handle 26 is integrally associated with housing 20 and movable handle 24 is movable relative to fixed handle 26. Movable handle 24 is ultimately connected to a drive assembly 70 that, together, mechanically cooperate to impart movement of one or both of jaw members 110, 120 of end effector assembly 100 relative to the other between a spaced-apart position and an approximated position to grasp tissue therebetween. As shown in FIG. 1, movable handle 24 is initially spaced-apart from fixed handle 26 and, correspondingly, jaw members 110, 120 are disposed in the spaced-apart position (see also FIG. 2A). Movable handle 24 is movable from this initial position to one or more compressed positions corresponding to one or more approximated positions of jaw members 110, 120 (see FIG. 2B).

Drive assembly 70 may be configured to regulate the clamping force applied to tissue grasped between surfaces 112, 122 of jaw members 110, 120, respectively. More specifically, handle assembly 22 and/or latching assembly 27, in conjunction with drive assembly 70, may be configured such that jaw members 110, 120 impart a specific clamping force or clamping force within a specific clamping force range to tissue grasped between surfaces 112, 122 of jaw members 110, 120, respectively. This may be achieved manually, e.g., via moving movable handle 24 from the initial position to a specific compressed position (or positions), e.g., a fully compressed position; via mechanical latching, e.g., wherein a latch assembly 27 is configured to latch movable handle 24 in a specific position (or positions); via a powered actuator with feedback-based control, e.g., via driving or reversing a motor-controlled actuator to a specific position (or positions); and/or via any other suitable mechanism. Drive assembly 70, in any of the configurations detailed above or any other suitable configuration, may include one or more passive regulating components, e.g., springs, resilient features, etc., and/or active regulating components, e.g., motor(s), manual drives, etc.

Suitable mechanisms for use as or in conjunction with drive assembly 70 for clamping force control include those described in U.S. Pat. Nos. 5,776,130; 7,766,910; 7,771,426; and 8,226,650; and/or U.S. Patent Application Pub. Nos. 2009/0292283; 2012/0172873; and 2012/0184988, the entire contents of all of which are hereby incorporated by reference herein. Other suitable mechanisms for applying a specific clamping force or clamping force within a specific clamping force range to tissue grasped between jaw members 110, 120 may also be provided. With tissue grasped between jaw members 110, 120 under the specific clamping force or clamping force within a specific clamping force range, energy may be supplied to either or both tissue contacting surfaces 112, 122 of jaw members 110, 120, respectively, to seal tissue, e.g., via activation of activation switch 30.

The jaw clamping force, measured at a midpoint along the lengths of jaw members 110, 120, may be in a range of (or the jaw force range may be) from about 7.0 lbf to about 11.0 lbf; in other aspects from about 8.0 lbf to about 10.0 lbf; and, in still other aspects, from about 8.5 lbf to about 9.5 lbf.

Latching assembly 27 may be provided for selectively locking movable handle 24 relative to fixed handle 26 at various positions between the initial position and the compressed position(s) to correspondingly lock jaw members 110, 120 at various different positions during pivoting, e.g., the one or more approximated positions. Rotating assembly 28 is rotatable in either direction to similarly rotate shaft 12 and end effector assembly 100 relative to housing 20.

Referring also to FIGS. 2A and 2B, end effector assembly 100 is shown attached at the distal end portion 16 of shaft 12 and includes opposing jaw members 110 and 120. Each jaw member 110, 120 includes an electrically conductive tissue contacting surface 112, 122, respectively, that cooperate to grasp tissue therebetween, e.g., in the one or more approximated positions of jaw members 110, 120, and to facilitate sealing the grasped tissue via conducting the energy from generator 40 therebetween. More specifically, tissue contacting surfaces 112, 122 are electrically coupled to generator 40, e.g., via wires 56a, 56b, and are configured to be energized to different potentials to enable the conduction of Radio Frequency (RF) electrosurgical energy provided by generator 40 between tissue contacting surfaces 112, 122 and through tissue grasped therebetween to seal tissue. Tissue contacting surfaces 112, 122 may be defined by electrically conductive plates secured to jaw members 110, 120, may be defined by surfaces of jaw members 110, 120 themselves, may be formed via the deposition of material onto jaw members 110, 120, or may be defined and/or formed in any other suitable manner.

Either or both jaw members 110, 120 may further include one or more stop members 124 (FIG. 2A) disposed on or otherwise associated with either or both tissue-contacting surface 112, 122 to maintain a minimum gap distance between tissue contacting surfaces 112, 122 when jaw members 110, 120 are disposed in a fully approximated position, thus inhibiting electrical shorting. Stop members 124 may be insulative, partly insulative, and/or electrically isolated from either or both tissue contacting surfaces 112, 122. In aspects, in the approximated position of jaw members 110, 120, it is desirable to maintain a gap distance within a suitable gap distance range to ensure consistent and effective tissue sealing. The gap distance may be controlled by stop members 124, movable handle 24, latching assembly 27, and/or drive assembly 70, and, in aspects, may be from about 0.001 inches to about 0.010 inches; in other aspects from about 0.001 inches to about 0.008 inches; and, in still other aspects form about 0.001 inches to about 0.006 inches. Other suitable gap distance ranges are also contemplated. The gap distance may be determined as the maximum gap distance between the tissue contacting surfaces 112, 122 at any point therealong.

An activation switch 30 is disposed on housing 20 and is coupled between or otherwise to generator 40 and/or tissue-contacting surfaces 112, 122 via wire 56c. Activation switch 30 is selectively activatable to initiate the supply of energy from generator 40 to tissue contacting surfaces 112, 122 of jaw members 110, 120, respectively, of end effector assembly 100. More specifically, depression of activation switch 30 is recognized, e.g., as a resistance drop, by generator 40 to signal to generator 40 to initiate tissue sealing, e.g., to supply energy to jaw members 110, 120.

End effector assembly 100 is designed as a bilateral assembly, e.g., wherein both jaw member 110 and jaw member 120 are movable about a pivot 19 relative to one another and to shaft 12. However, end effector assembly 100 may alternatively be configured as a unilateral assembly, e.g., wherein one of the jaw members 110, 120 is fixed relative to shaft 12 and the other jaw member 110, 120 is movable about pivot 19 relative to shaft 12 and the fixed jaw member.

In some configurations, a knife assembly (not shown) is disposed within shaft 12 and a knife channel 115 is defined within one or both jaw members 110, 120 to permit reciprocation of a knife blade (not shown) therethrough, e.g., via actuation of trigger assembly 25, to mechanically cut tissue grasped between jaw members 110, 120. In aspects, the knife blade is energizable to enable dynamic energy-based tissue cutting. Alternatively, end effector assembly 100 may include a static energy-based tissue cutter (not shown), e.g., disposed one or within one of the jaw members 110, 120. The energy-based cutter, whether static or dynamic, may be configured to supply any suitable energy, e.g., RF, microwave, infrared, light, ultrasonic, thermal, etc., to tissue for energy-based tissue cutting. Energy activation for tissue cutting may be initiated via trigger assembly 25, automatically after tissue sealing, via a different (or further) activation of switch 30, via a separate actuation button, via a foot switch (not shown), or in any other suitable manner.

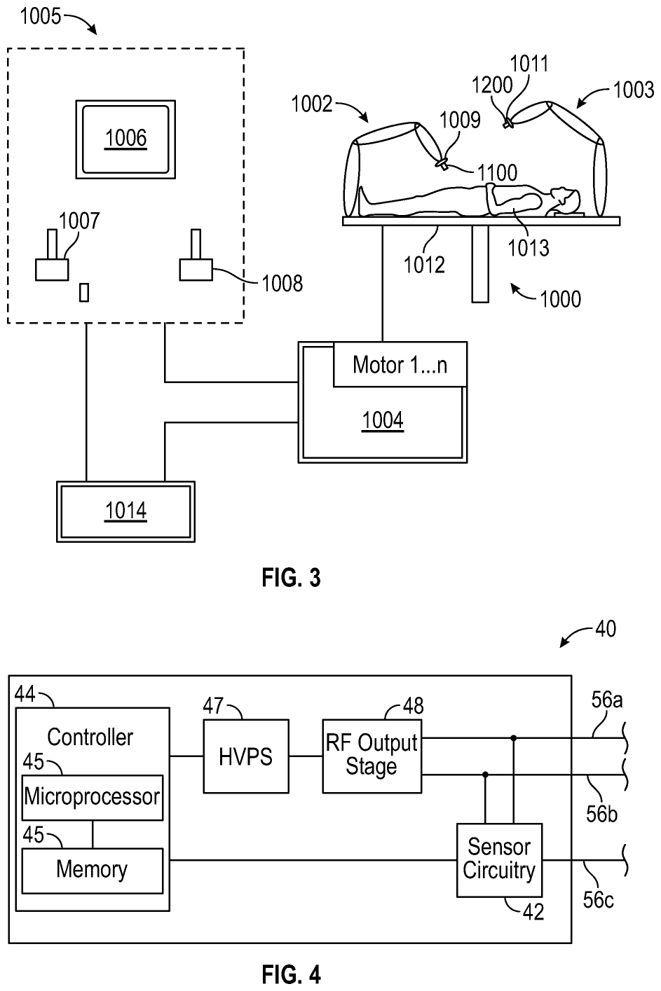

Turning to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in an operating mode. Robotic surgical instrument 1000 may be configured for use on a patient 1013 lying on a patient table 1012 to be treated in a minimally invasive manner. Robotic surgical instrument 1000 may further include or be capable of accessing a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100, for example, may be similar to and include any of the features of end effector assembly 100 (FIGS. 1-2B) and, together with robot arm 1002, functions similarly as detailed above with respect to forceps 10 except in a robotically-actuated and controlled manner. Other suitable end effector assemblies for coupling to attaching device 1009 are also contemplated. End effector assembly 1200 may be any end effector assembly, e.g., a surgical camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

With reference to FIG. 4, generator 40 may be configured for use with forceps 10 (FIG. 1), robotic surgical system 1000 (FIG. 3), and/or any other suitable surgical instrument or system. Generator 40 includes sensor circuitry 42, a controller 44, a high voltage DC power supply (“HVPS”) 47 and an RF output stage 48. HVPS 47 provides high voltage DC power to RF output stage 48 which converts the high voltage DC power into RF energy for delivery to the end effector assembly, e.g., tissue-contacting surfaces 112, 122 of jaw members 110, 120, respectively, of end effector assembly 100 (FIGS. 1-2B). In particular, RF output stage 48 generates sinusoidal waveforms of high frequency RF energy. RF output stage 48 is configured to generate a plurality of waveforms having various duty cycles, peak voltages, crest factors, and other parameters, depending on a particular mode of operation.

Controller 44 includes a microprocessor 45 operably connected to a memory 46 which may be volatile type memory (e.g., RAM) and/or non-volatile type memory (e.g., flash media, disk media, etc.). Microprocessor 45 is operably connected to HVPS 47 and/or RF output stage 48 allowing microprocessor 45 to control the output of generator 40, e.g., in accordance with feedback received from sensor circuitry 42. Sensor circuitry 42 is operably coupled to wires 56a, 56b, which supply energy to/from tissue-contacting surfaces 112, 122 (FIGS. 1-2B). From wires 56a, 56b and, more specifically, the signals transmitted therealong, sensor circuitry 42 may determine one or more parameters, e.g., tissue impedance, output current and/or voltage, etc. Sensor circuitry 42 provides feedback, e.g., based on the sensed parameter(s), to controller 44 which, in turn, selects an energy-delivery algorithm, modifies an energy-delivery algorithm, and/or adjusts energy-delivery parameters based thereon. Sensor circuitry 42 or controller 44 may also monitor wire 56c to determine activation (and/or deactivation) of switch 30 (FIG. 1) to, in response thereto, initiate (or terminate) the supply of energy based thereon.

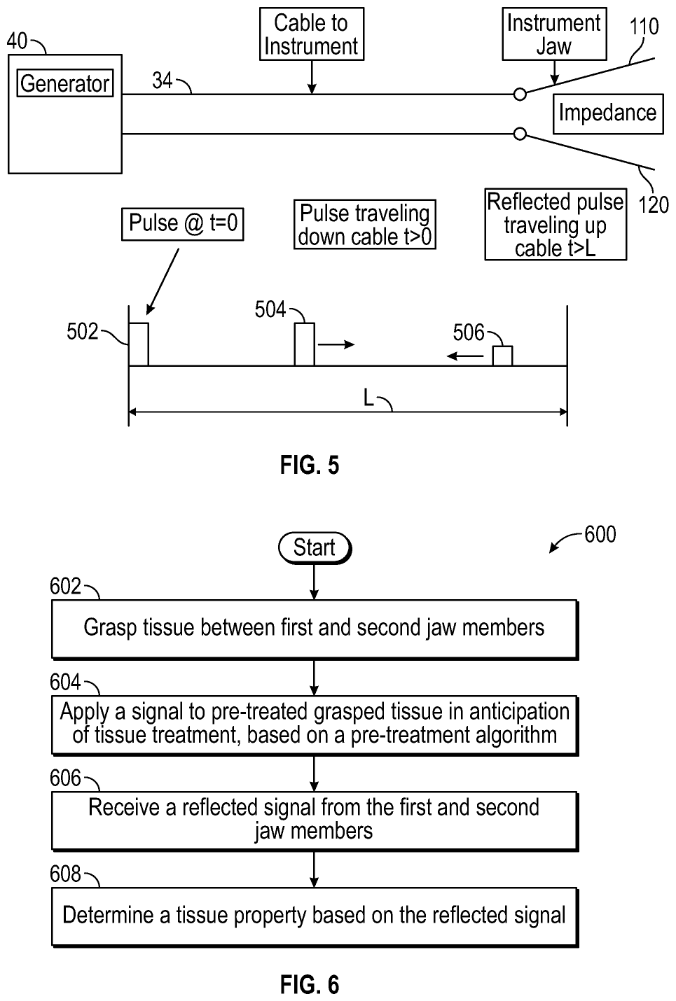

Referring to FIG. 5, a schematic illustration of system 2 (FIG. 1) illustrates the generator 40 connected to jaw members 110, 120 by a cable 34. A signal, such as a pulse 502, is transmitted on the cable 34. The signal travels at a velocity that is a function of the characteristic impedance of the cable 34. For example, the characteristic impedance of the cable may be 50 ohms. However, any suitable characteristic impedance may be used. The time it takes for the signal to travel the length of the cable (e.g., about 150 nS for a 10 foot cable) is the velocity per unit length divided by the physical length of the cable. The time to travel the length of cable is called the electrical length “L.” If the signal (i.e., pulse 502) is shorter in duration than the electrical length of the cable “L,” the reflection will return to the generator 40 after the generator 40 has completed transmission. This allows for a time discrimination scheme where a receiver at the generator output is turned off during the time the signal is being transmitted (i.e., the pulse width) and turned on at the time required for the signal to travel the length of the cable 34 and return.

To measure a property of tissue (and/or other matter), such as impedance, between the jaw members 110, 120, a short duration pulse may be transmitted down the instrument cable 34 and the returned reflected signal is measured. Although impedance is used as an example, other properties are also contemplated.

A difference between the characteristic impedance of tissue (and/or other matter) that is grasped and the characteristic impedance of the cable 34 and the generator 40, will cause a reflection of the signal (i.e., pulse 502). The impedance between the jaw members 110, 120 will reflect some of the signal (i.e., reflected pulse 506) back to the generator 40. The amount of the reflection is a function of the impedance between the jaw members 110, 120 and the characteristic impedance of the cable 34. The characteristic impedance of the cable 34 is relatively a constant, so changes in the amplitude of the reflected signal are directly related to the impedance between the jaw members 110, 120. In aspects, a Voltage Standing Wave Ratio (VSWR) mismatch between the grasped tissue and the characteristic impedance of the system (e.g., 50 ohms) may be determined from the amplitude of the reflected signal. The resultant VSWR may be used to determine tissue impedance (and thereby, tissue type, tissue thickness, tissue moisture, etc.) and/or other conditions in and around the jaw members 110, 120, e.g., whether the jaw members 110, 120 are submerged in saline, whether a conductive material is in the vicinity, whether an open or short circuit condition exists, etc.

The impedance (and/or other characteristics) may be derived from a time-domain voltage measurement of the amplitude of the reflected pulse. The impedance may also be derived from changes in frequency spectrum and/or a frequency domain measurement. In aspects, impedance, phase, and/or amplitude at more than one frequency of the return signal may be measured and used to determine a tissue property. More specifically, measuring impedance, phase, and/or amplitude at multiple frequencies of the return signal provides a feedback spectrum, e.g., of impedance, allowing for tissue properties to be ascertained based on not only singular impedance measurements but also relative impedance measurements at different frequencies. The same can be accomplished with phase, amplitude, and/or time.

FIG. 6 shows a flow diagram for an exemplary computer-implemented method for sealing tissue. Although the steps of FIG. 6 are shown in a particular order, the steps need not all be performed in the specified order, and certain steps can be performed in another order. In various aspects, the operations of FIG. 6 may be performed all or in part by the controller 44 of the generator 40 of FIG. 4. In aspects, the operations of FIG. 6 may be performed all or in part by another device, for example, a mobile device and/or a client computer system. These variations are contemplated to be within the scope of the present disclosure.

Initially, at step 602, tissue is grasped between first and second jaw members 110, 120 (FIG. 5).

Next, at step 604, the controller 44 applies a signal to grasped tissue in anticipation of tissue treatment. The signal is based on a tissue sense algorithm. For example, the signal may include a short pulse that has approximately a 500 mV amplitude and a pulse duration of about 100 nS. The signal is configured to reflect from the first and second jaw members 110, 120 back through the cable 34. The tissue sense algorithm may include an amplitude of the signal, a duration of the signal, and/or a repetition rate of the signal.

Next, at step 606, the controller 44 receives a reflected signal from the first and second jaw members 110, 120. For example, the reflected signal may have an amplitude of approximately 30 mV.

Next, at step 608, the controller 44 determines a tissue property and/or other condition based on the reflected signal. For example, the controller 44 may determine that the tissue is a particular type of tissue such as vascular, muscle, organ, etc., based on the reflected signal. The determined tissue type may be used to select and/or adjust a tissue sealing algorithm or to allow or prevent the subsequent application of energy. An impedance of the tissue (or impedance spectrum of the tissue across multiple frequencies of the reflected signal) may be utilized to determine the tissue type.

A delay time and/or a voltage of the reflected signal may additionally or alternatively facilitate determining the tissue property, e.g., tissue type. In aspects, other tissue properties can be determined based on the impedance of the tissue (or impedance spectrum of the tissue across multiple frequencies of the reflected signal) such as, for example, tissue thickness, tissue moisture content, tissue conductivity, etc.

The controller 44 may determine the tissue property based on determining a frequency content of the reflected signal, and then determining a tissue property based on the determined frequency content of the reflected signal. A fast Fourier transform (FFT) may be performed on the reflected signal (e.g., the reflected pulse). An ideal short pulse has an infinite series of harmonics at equal amplitude. In practice, due to a rising edge of a generated pulse and due to various parasitic capacitances and/or inductances, the harmonic series is not infinite and attenuates as it goes up in frequency. For a known short pulse with a known duration, an FFT may be performed to determine an initial frequency domain response. The controller 44 may perform an FFT on the received reflected signal to determine a frequency domain response for the reflected pulse. The controller 44 may compare the initial frequency domain response and the reflected frequency domain response and determine a tissue property based on the comparison.

In aspects, the controller 44 may adjust a tissue sealing algorithm based on the determined tissue property. More detailed tissue sealing algorithms suitable for use in accordance with the present disclosure can be found in, for example, U.S. Pat. No. 8,920,421, titled “SYSTEM AND METHOD FOR TISSUE SEALING” and filed as U.S. patent application Ser. No. 12/995,042 on Nov. 29, 2010, the entire contents of which are hereby incorporated herein by reference; and U.S. Pat. No. 8,147,485, titled “SYSTEM AND METHOD FOR TISSUE SEALING” and filed as U.S. patent application Ser. No. 12/391,036 on Feb. 23, 2009, the entire contents of which are hereby incorporated herein by reference.

In still other aspects, the tissue sense algorithm may additionally or alternatively be performed during and/or after the tissue sealing algorithm.

In aspects, the controller 44 may apply energy to the grasped tissue in accordance with a tissue sealing algorithm to seal the grasped tissue. The tissue sealing algorithm may be independent of the tissue sense algorithm or the tissue sense algorithm may be integrated into the tissue sealing algorithm. In aspects, the tissue sense algorithm is controlled in a first manner and the tissue sealing algorithm is controlled in a second, different manner.

In aspects, the controller 44 may apply the signal during treatment of tissue to determine changes in the tissue property. At least a portion of the tissue sealing algorithm may adjust energy output to track an impedance versus time trajectory.

In aspects, the controller 44 may determine whether the tissue sense algorithm is complete before applying energy to the grasped tissue in accordance with the tissue sealing algorithm.

After applying energy to the grasped tissue in accordance with the tissue sense algorithm and before applying the energy to the grasped tissue in accordance with the tissue sealing algorithm, the controller 44 may implement a delay period where no energy is applied.

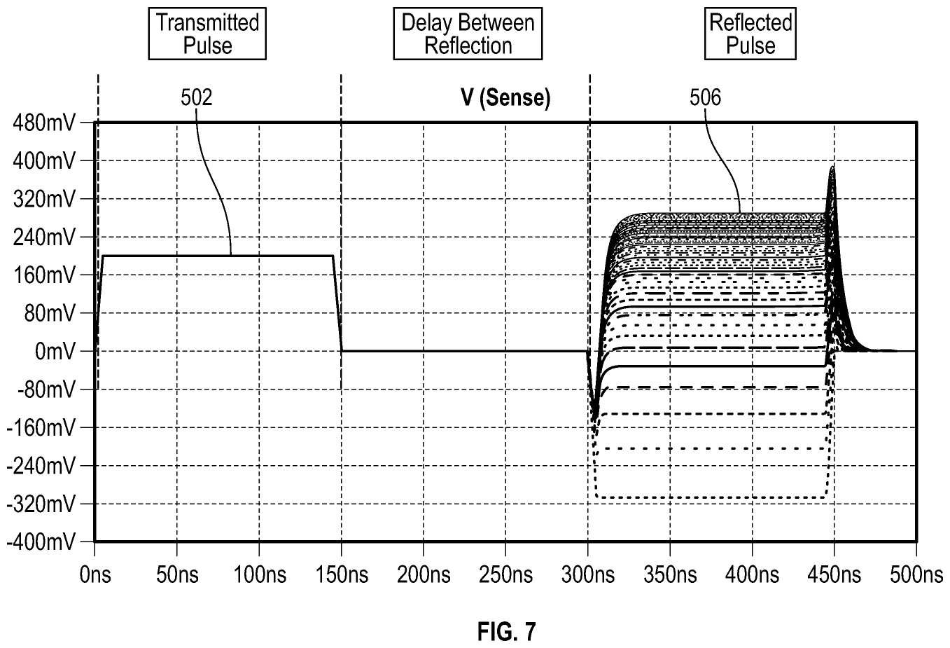

FIG. 7 shows a simulation of a signal (i.e., a pulse 502) with a 150 nS duration and a 150 nS delay between the reflection and the measured reflected pulse 506. An impedance of the load that the pulse is reflected off of is varied from 1 ohm to 1000 ohms. The simulation demonstrates how the returned pulse's amplitude changes with impedance changes.

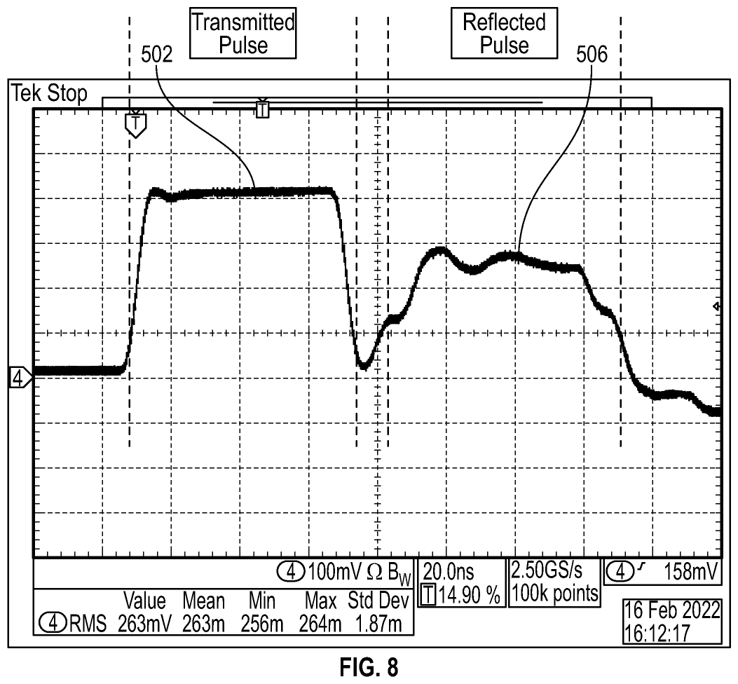

Referring to FIG. 8, an oscilloscope trace showing a transmitted signal (i.e., pulse 502) and a reflected signal (i.e., reflected pulse 506) with the first and second jaw members 110, 120 of FIG. 5 in an open position and not grasping tissue is provided. As can be seen in the oscilloscope trace, the applied signal is a pulse of about 60 nS in duration at an amplitude of about 400 mV peak. The length of the cable provides a delay of about 10 nS. With an “ideal” open circuit, the reflected pulse 506 would be equal in amplitude to the transmitted pulse 502. The observed reflected pulse 506 shows that the first and second jaw members 110, 120 of FIG. 5 have an impedance other than the characteristic impedance of 50 ohms. Thus, in addition to detecting tissue properties, other conditions associated with the first and second jaw members 110, 120 such as, for example, an open circuit condition, can be detected. Likewise, it can be determined whether a sufficient volume of tissue is grasped between the first and second jaw members 110, 120 or if a minimal tissue volume, insufficient to establish a proper tissue seal, is grasped. Further still, the presence of other materials, e.g., bone, staples, other instrumentation, etc. can also be detected.

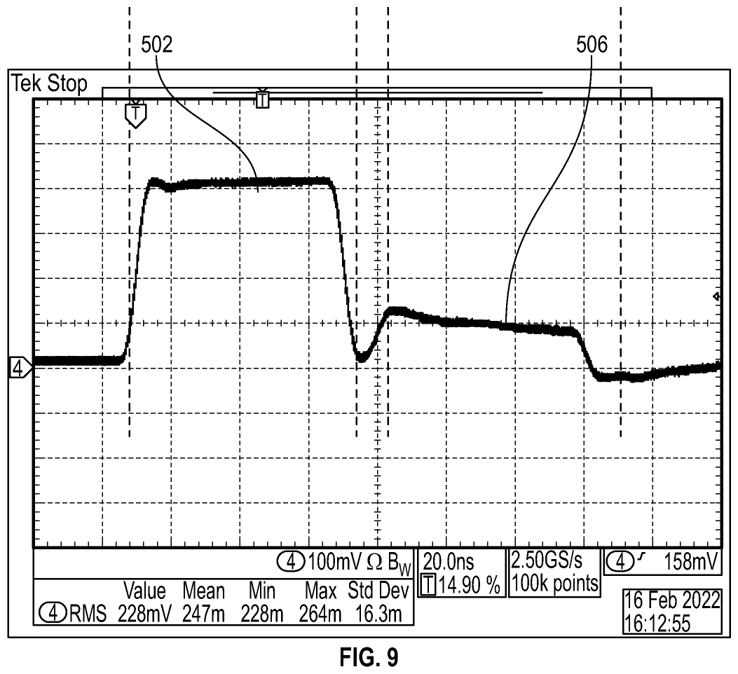

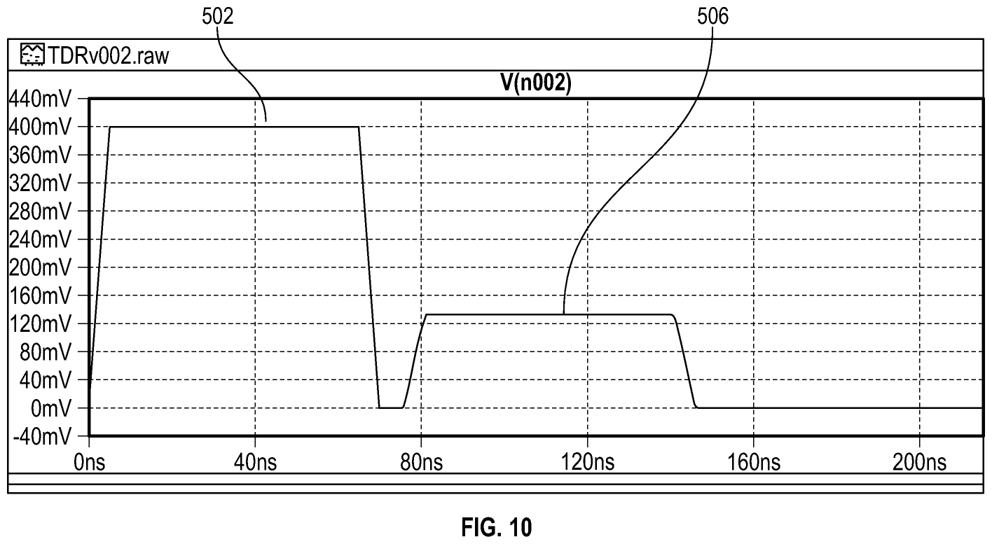

FIG. 9 shows an oscilloscope trace showing a transmitted pulse 502 and a reflected pulse 506 into an impedance of approximately 100 ohms. FIG. 10 shows a simulation of an ideal reflected pulse from a 100 ohm load. As can be seen from the oscilloscope trace in FIG. 9, the reflected pulse 506 amplitude is close to the simulated theoretical amplitude of about 138 mV peak of FIG. 10.

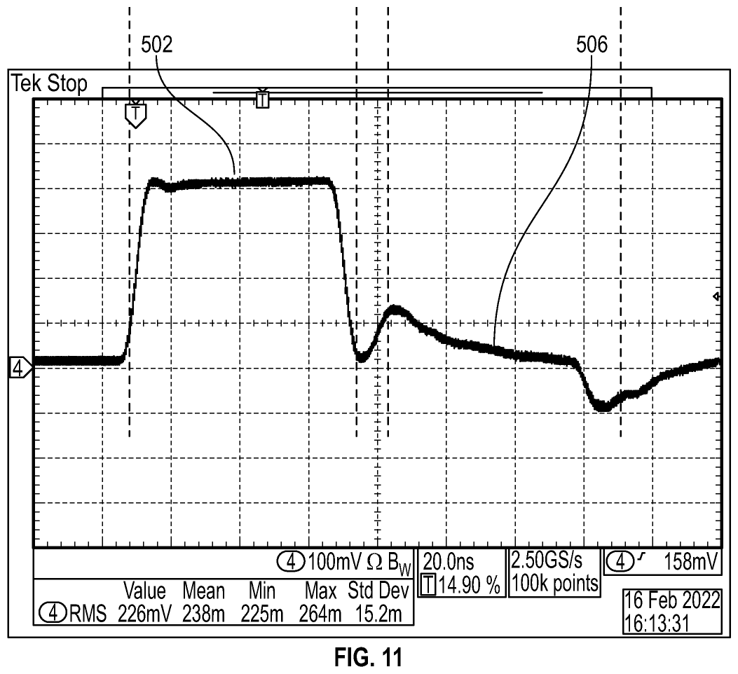

FIG. 11 shows an oscilloscope trace showing the transmitted pulse 502 and reflected pulse 506 with first and second jaw members 110, 120 of FIG. 5 grasping a load of approximately 50 ohms. From the oscilloscope trace, the amplitude of the reflected pulse 506 is close to the theoretical amplitude of about zero volts peak for a 50 ohm system. The characteristic impedance of the coaxial cable is 50 ohms so there should be almost zero reflected signal. The actual reflected pulse 506 trace shows some non-zero voltage which is due to parasitic capacitance and inductance of the first and second jaw members 110, 120 of FIG. 5. The voltage reaches close to zero volts at the end of the pulse.

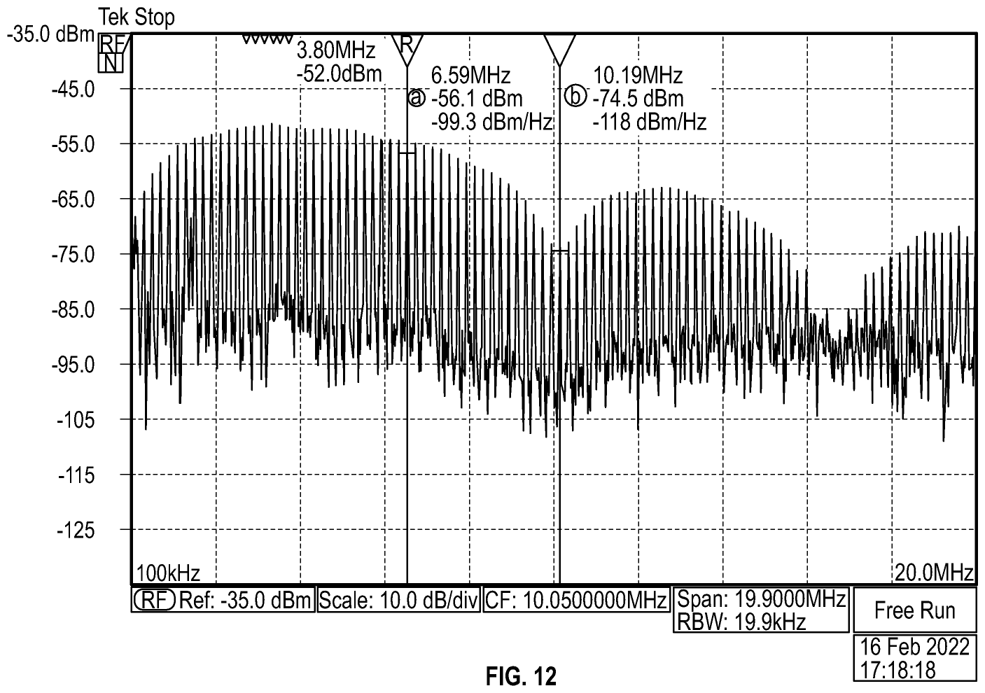

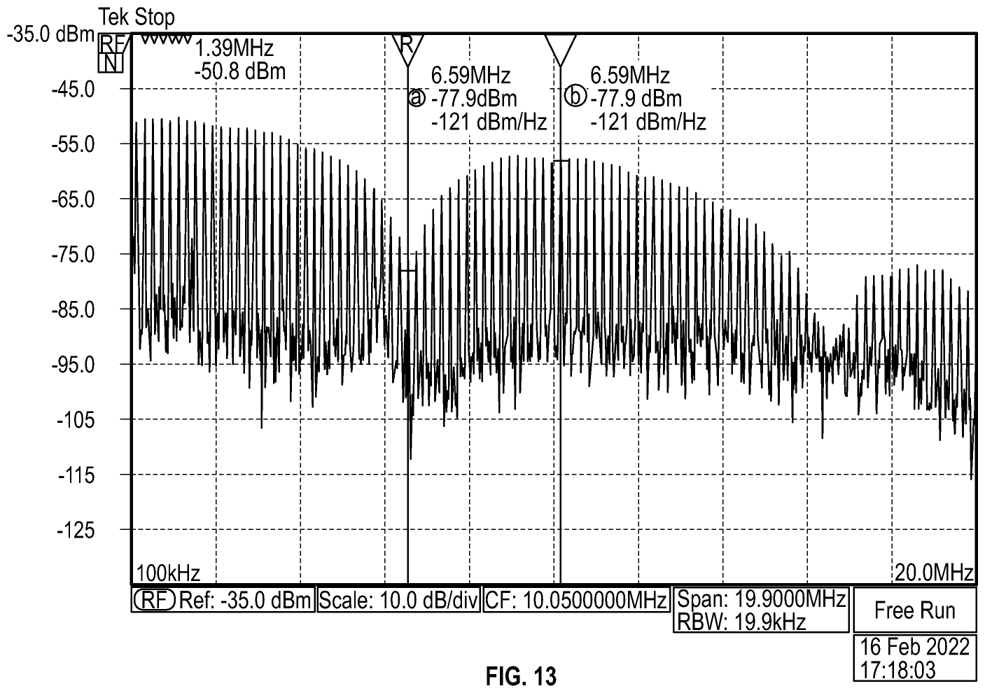

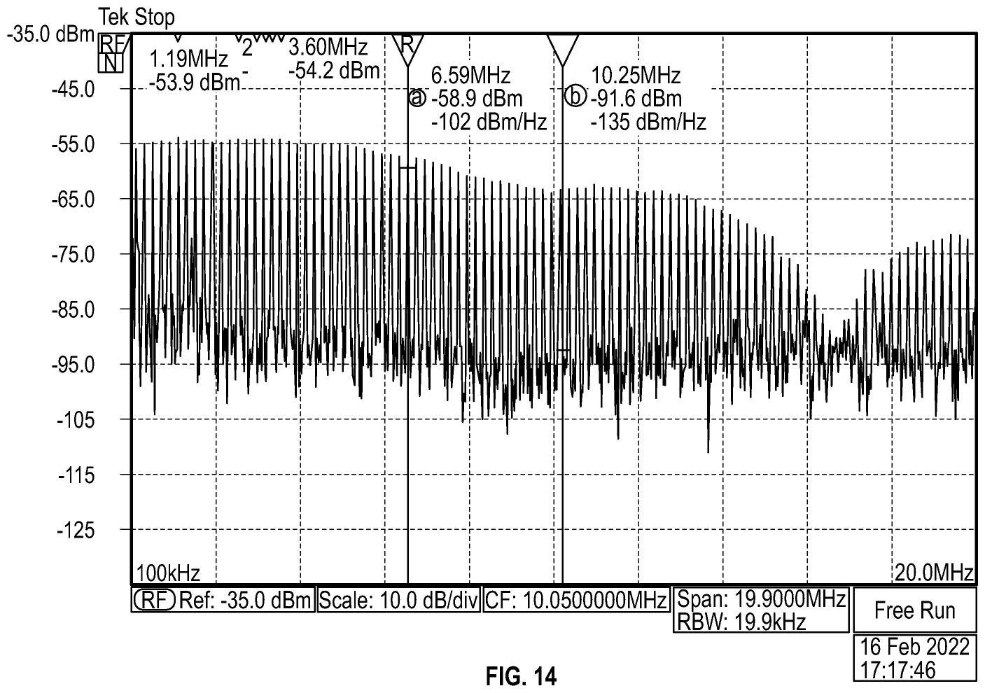

Referring to FIGS. 12-14, shows frequency domain plots of the frequency domain response of the transmitted pulse 502 and reflected pulse 504 for three exemplary impedance conditions of a short (FIG. 12), an open (FIG. 13), and a 50 ohm impedance (FIG. 14) between the first and second jaw members 110, 120. A short pulse 502, approximately 60 nS in duration with a very low amplitude of about 40 mV peak is applied to the first and second jaw members 110, 120 of FIG. 5 and the frequency spectrum is shown.

The results show the spectrum is different for the different conditions. Two frequencies were chosen to show the changes. Markers were placed at about 6.19 MHz and about 10.19 MHz. The amplitude at these two frequencies could be seen to change as summarized in the table below.

The results show the spectrum does change with impedance showing that frequency domain or frequency response to a short pulse may be used to determine impedance changes and differentiate tissue types and/or other tissue properties.

FIG. 12 is a trace showing the frequency spectrum with a short circuit between first and second jaw members 110, 120 of FIG. 5. FIG. 13 is a trace showing the frequency spectrum with an open circuit between first and second jaw members 110, 120. FIG. 14 is a trace showing the frequency spectrum with the first and second jaw members 110, 120 grasping a load of about 50 ohms.

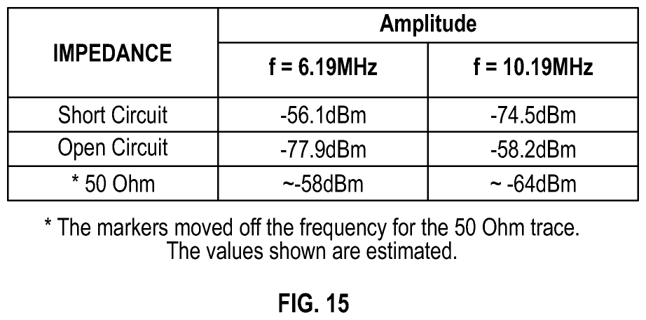

FIG. 15 is a table that shows the impedance vs. amplitude at different frequencies for a return pulse for the load conditions shown in FIGS. 12-14. For example, for the short circuit load shown in FIG. 12, at about 6.19 MHz, an amplitude of about −56 dBm is measured. At a frequency of about 10.19 MHz, an amplitude of about −74 dBm is measured. For the open circuit load of FIG. 13, at about 6.19 MHz, an amplitude of about −78 dBm is measured. At a frequency of about 10.19 MHz, an amplitude of about −58 dBm is measured. For the 50 ohm load of FIG. 14, at about 6.19 MHz, an amplitude of about −58 dBm is measured. At a frequency of about 10.19 MHz, an amplitude of about −64 dBm is measured.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A method of treating tissue, comprising:
grasping tissue between first and second jaw members;
applying a signal to the grasped tissue in anticipation of tissue treatment based on a tissue sense algorithm;
receive a reflected signal from the first and second jaw members at a plurality of frequencies; and
determining a tissue property or a condition at the first and second jaw members based on the reflected signal.

2. The method according to claim 1, further comprising adjusting a tissue sealing algorithm based on the determined tissue property.

3. The method according to claim 1, further comprising applying energy to the grasped tissue in accordance with a tissue sealing algorithm to seal the grasped tissue, wherein the tissue sealing algorithm is independent of the tissue sense algorithm.

4. The method according to claim 1, wherein the tissue sense algorithm is controlled in a first manner and the tissue sealing algorithm is controlled in a second, different manner.

5. The method according to claim 1, wherein a delay time and/or a voltage of the reflected signal at the plurality of frequencies is utilized to determine the tissue property.

6. The method according to claim 5, wherein the tissue property is impedance at each of the plurality of frequencies.

7. The method according to claim 1, wherein at least a portion of the tissue sealing algorithm adjusts energy output to track an impedance versus time trajectory.

8. The method according to claim 1, wherein the condition at the first and second jaw members is one of an open circuit condition or a short circuit condition.

9. The method according to claim 1, wherein determining the tissue property includes:
determining a frequency content of the reflected signal; and
determining a tissue property based on the determined frequency content of the reflected signal.

10. The method according to claim 1, further comprising, after applying energy to the grasped tissue in accordance with the tissue sense algorithm and before applying the energy to the grasped tissue in accordance with the tissue sealing algorithm, implementing a delay period where no energy is applied.

11. An electrosurgical system for treating tissue, comprising:
electrosurgical forceps including first and second jaw members; and
an electrosurgical generator including:
a processor; and
a memory, including instructions stored thereon, which, when executed by the processor cause the system to:
apply a signal to tissue that is grasped between the first and second jaw members in anticipation of tissue treatment, based on a tissue sense algorithm;
receive a reflected signal from the first and second jaw members at a plurality of frequencies; and
determine a tissue property or a condition at the first and second jaw members based on the reflected signal.

12. The electrosurgical system according to claim 11, wherein the instructions, when executed by the processor, further cause the system to adjust a tissue sealing algorithm based on the determined tissue property.

13. The electrosurgical system according to claim 11, wherein the instructions, when executed by the processor, further cause the system to apply energy to the pre-treated, grasped tissue in accordance with a tissue sealing algorithm to seal the pre-treated grasped tissue, wherein the tissue sealing algorithm is independent of the pre-treatment algorithm.

14. The electrosurgical system according to claim 11, wherein the instructions, when executed by the processor, further cause the system to control the tissue sense algorithm in a first manner and the tissue sealing algorithm is controlled in a second, different manner.

15. The electrosurgical system according to claim 11, wherein a delay time and/or a voltage of the reflected signal at the plurality of frequencies is utilized to determine the tissue property.

16. The electrosurgical system according to claim 15, wherein the tissue property is impedance at each of the plurality of frequencies.

17. The electrosurgical system according to claim 11, wherein at least a portion of the tissue sealing algorithm adjusts energy output to track an impedance versus time trajectory.

18. The electrosurgical system according to claim 11, wherein the condition at the first and second jaw members is one of an open circuit condition or a short circuit condition.

19. The electrosurgical system according to claim 11, wherein when determining the tissue property, the instructions, when executed by the processor, further cause the system to:
determine a frequency content of the reflected signal; and
determine a tissue property based on the determined frequency content of the reflected signal.

20. A non-transitory computer-readable medium storing instructions which, when executed by a processor, cause the processor to perform a computer-implemented method of treating tissue, comprising:
grasping tissue between first and second jaw members;
applying a signal to the grasped tissue in anticipation of tissue treatment based on a tissue sense algorithm;
receive a reflected signal from the first and second jaw members at a plurality of frequencies; and
determining a tissue property or a condition at the first and second jaw members based on the reflected signal.
